(19) United States
(12) Patent Application Publication
(10) Pub. No.: US 2015/0125839 A1
(43) Pub. Date: May 7, 2015

(54) WIRELESS COMMUNICATION FOR PRESSURE SENSOR READINGS

(71) Applicant: TILLGES TECHNOLOGIES LLC, Maplewood, MN (US)

(72) Inventors: Robert James Tillges, White Bear Lake, MN (US); Michael Robert Tillges, White Bear Township, MN (US); Steven James Tillges, Centerville, MN (US)

(21) Appl. No.: 14/369,117

(22) PCT Filed: Jul. 27, 2012
(86) PCT No.: PCT/US2012/048506

§ 371 Date: Jun. 26, 2014

(51) Int. Cl. A61B 5/00; A61B 5/11; G09B 5/00; A61B 5/103

(52) U.S. Cl. CPC A61B 5/0022; A61B 5/0004; A61B 5/1038; A61B 5/112; G09B 5/00
USPC 434/262; 600/592

(57) ABSTRACT

Aspects of the instant disclosure relate to an orthosis pressure sensing device and methods of use. The pressure sensing device utilizes a Control unit connected to at least one pressure sensing arrangement placed in proximity to an orthosis. The control unit collects data from the pressure sensing arrangement, and wirelessly communicates data to various remote devices, which may be useful for improving a patient's gait, wound recovery or overall function. Each pressure sensor arrangement has a pressure sensor designed for selective placement at multiple locations of interest that include different contact points between an orthosis and a patient.

### OVERVIEW

The aspects of the present disclosure relate generally to wireless communications of pressure sensor readings and to systems, methods and devices for facilitating use of pressure sensors for orthotics.

A functional lower-limb orthosis includes orthopedic devices that are designed to promote structural integrity of the joints of the foot or lower limb. The orthosis resists ground reaction forces, which can otherwise cause abnormal skeletal motion (e.g., during the stance phase of gait). A lower-limb orthosis can be used on a portion of the lower body to provide various benefits, such as support, improved gait, off-loading of weight from around a wound and correction of an orthopedic problem, deformity or functional impairment. Such a lower-limb orthosis can be designed to interface with a shoe, foot, ankle, knee, hip or combinations thereof. Orthosis can function as a static device or as a dynamic device. Static orthoses are often rigid and can be used to support weakened or paralyzed body parts to maintain them in a particular position. A dynamic orthosis can be used to facilitate body motion by improving limb function.

A lower-limb orthosis is often used for the specific management of a particular disorder. The orthotic joints can be aligned to correspond to the approximate anatomic joints. Many orthoses use a three-point system to ensure proper positioning of the lower limb inside the orthosis. The patient may benefit from an orthosis that is simple, lightweight, strong, durable, and cosmetically acceptable. Considerations for an orthotic prescription can include the possible need for a three-point pressure control system, static or dynamic stabilization, flexible material, and tissue tolerance to compression and shear force.

Despite improvements in orthotic technology and methodology, a number of problems and challenges still exist. For instance, each patient represents a unique combination of relevant characteristics including, but not necessarily limited to, limb morphology, patient height, patient weight, gait length, patient musculature/strength, and the proper fit for one or more disorders or other complications (e.g., diabetes). These and other characteristics can cause significant differences in the effectiveness of an orthosis. The patient may also have difficulties in learning how to properly use the orthosis.

Even when special care is given to selecting and fitting an orthosis, as well as to training a patient in proper use, adjustments are often necessary. Sometimes an adjustment becomes necessary because of a wound or sore caused by excessive skin abrasion. For example, patients with neuropathy whom lack the sense of feeling experiencing the pain caused by a wound may hinder an assessment of the amount of pressure between the orthosis and either the wound or the surrounding area.

### SUMMARY

Aspects of the present disclosure are directed to orthosis pressure sensing devices, and methods of using, that address challenges including those discussed herein, and that are applicable to a variety of applications. These and other aspects of the present invention are exemplified in a number of implementations and applications, some of which are shown in the figures and characterized in the claims section that follows.

Consistent with various embodiments, aspects of the instant disclosure are directed towards orthosis pressure sensing devices, and methods of using the devices. The orthosis pressure sensing devices include a control unit and one or more pressure sensor arrangements connected to the control unit. The control unit includes an output port that receives pressure data from the pressure sensor (included in the pressure sensor arrangements). A wireless interface circuit is provided in the control unit in order to communicate with remote devices using wireless communications. The control unit also includes a processing circuit that is configured to detect a presence of a wireless device running a software application. The detection is responsive to data received from the wireless interface circuit. The processing circuit also establishes communications with the detected wireless device by exchanging data with the software application (using the wireless interface circuit). Pressure data received from the input port (according to a wireless transmission protocol used by the software application) is formatted by the processing circuit, and the data is transmitted, using the wireless interface circuit, to the software application.

The of the pressure sensor arrangements of the orthosis pressure sensing devices can include an output port designed for insertion into and removal from the input port, which creates a communication link to the input port when inserted. The pressure sensor arrangements can also include a pressure sensor. The pressure sensor is designed for selective placement at multiple locations of interest, including different contact points between an orthosis and a patient. The pressure sensor arrangement can provide a communication line that carries the pressure data from the pressure sensor to the output interface.

Additionally, aspects of the instant disclosure are directed toward methods for sensing pressure between an orthosis and tissue of a patient. In these methods, the shape and size of a patient wound are assessed in order to select a pressure sensor based upon a correlation between a pattern of an active pressure sensing area for the pressure sensor and the assessed shape and size of the patient wound. The selected pressure sensor is fixed to a location proximal to the patient wound to allow pressure between the orthosis and patient tissue to be sensed by the pressure sensor, thereby minimizing further agitation of the patient wound. The pressure sensor is communicatively coupled to a control unit. A wireless connection is established between the control unit and a software application running on a wireless communication device. The pressure data received from the pressure sensor is communicated to the software application using the wireless connection, and, using the software application, pressure data feedback.

The above summary is not intended to describe each illustrated embodiment or every implementation of the present invention.

### BRIEF DESCRIPTION OF THE DRAWINGS

The invention may be more completely understood in consideration of the following detailed description of various embodiments of the invention in connection with the accompanying drawings, in which:

FIG. 1 depicts an example diagram of an orthosis pressure sensing system, consistent with various embodiments of the present disclosure;

FIG. 2 shows a block diagram of an example control unit, consistent with embodiments of the present disclosure;

FIG. 3 shows various example sensor arrangements, consistent with various embodiments of the present disclosure;

FIG. 4 shows an example flowchart of the operation of an orthosis pressure sensing system, consistent with various embodiments of the present disclosure;

FIG. 5 shows placement of a device with an orthosis pressure sensing system, consistent with various embodiments of the present disclosure;

FIG. 6 shows a device and a control unit on an orthosis for measuring orientation changes during the gait of a patient wearing the orthosis, consistent with various embodiments of the present disclosure, and

FIG. 7 depicts a plurality of different gait phases and a corresponding pressure waveform, consistent with embodiments of the present disclosure.

While the invention is amenable to various modifications and alternative forms, specifics thereof have been shown by way of example in the drawings and will be described in detail. It should be understood, however, that the invention is not necessarily limited to the particular embodiments described. On the contrary, the intention is to cover all modifications, equivalents, and alternatives falling within the spirit and scope of the invention.

### DETAILED DESCRIPTION

Aspects of the present disclosure are directed to pressure sensing in various locations on an orthosis or on patient's body and to related approaches, their uses and systems for the same. While the present invention is not necessarily limited to such applications, various aspects of the invention may be appreciated through a discussion of various examples using this context.

Certain aspects of the present disclosure are directed toward a system or device for obtaining pressure measurements for a wide variety of orthoses, patients, interface devices, conditions, specialists and environments. One or more of the embodiments discussed herein can be particularly useful in connection with diverse environments and applications and also to adapt to changing parameters. One or more of the discussed embodiments can also be particularly useful for problems associated with various diverse applications; however, the embodiments are not necessarily limited to a particular application. Moreover, unless otherwise stated, the various embodiments can be used in combination with one another and/or for additional purposes beyond those expressly stated. The discussion herein includes a number of different embodiments that relate to flexible and diverse qualities of pressure sensing devices and methods.

Various aspects of the instant disclosure are directed towards an orthosis pressure sensing device. The orthosis pressure sensing device includes a control unit that has an input port designed to receive pressure data from a pressure sensor. The input port can be arranged with a number of different connectors (e.g., Universal Serial Bus (USB) compatible connectors and/or cords) in order to facilitate data transfer to and from the control unit and at least one pressure sensor. Additionally, the control unit is provided with at least one wireless interface in order to communicate with remote devices using wireless communication (e.g., Bluetooth®, WiFi, IEEE 802.xx or WiMax). The control unit also includes a processing circuit that is configured to detect the presence of a wireless device running a software application (such as an application for analyzing pressure data). This detection occurs in response to data received from the wireless interface circuit. The processing circuit can establish communications with the detected wireless device by exchanging data with the software application using the wireless interface circuit. The pressure data received from the input port of the control unit can then be formatted by the processing circuit according to the wireless transmission protocol used by the software application of the remote device. Using the wireless interface circuit, the formatted pressure is transmitted to the software application of the remote device.

The pressure data received by the control unit is provided by one or more pressure sensor arrangements of the orthosis pressure sensing device. Each pressure sensor arrangement includes an output port designed for insertion into and removal from the control unit's input port, which creates a communication link to the input port when inserted. Each pressure sensor arrangement has a pressure sensor designed for selective placement at multiple locations of interest that include different contact points between an orthosis and a patient. A communication line, included with each pressure sensor arrangement, carries pressure data from the pressure sensor to the output interface. Various aspects of the present disclosure provide for hot swapping (replacing while still powered up) sensors to and from the control unit's input port. This type of quick disconnect/reconnect capability can be facilitated by the use of USB or other connectors, which are designed for hot swapping environments.

Certain, more specific embodiments, can include multiple pressure arrangements. Each arrangement can include a corresponding and respective pressure sensor with different pressure sensing patterns, which can be selected relative to a size and shape of a patient wound. Patients using an orthosis often can have irritated skin or wounds that result from daily wear of the orthosis. The pressure sensors included in the orthosis pressure sensing device can also be provided with a pattern, such as an opening, to sense pressure between an orthosis and patient tissue surrounding a wound while reducing or avoiding contact with the patient wound. These pressure sensor arrangements, in various embodiments of the instant disclosure, include an attachment mechanism to facilitate placement of the pressure sensor to each of multiple different locations in the orthosis. In this manner, pressure sensor placements can be optimized and selected relative to a specific orthosis, which is fitted to a specific patient. In embodiments that include multiple pressure sensor arrangements, the control unit is provided with additional input ports that can each be designed to receive pressure data from corresponding and respective pressure sensors.

The processing circuit of the orthosis pressure sensing system, in certain embodiments, performs the formatting of the pressure data received from the input port and transmits the formatted pressure data in real time, relative to receipt of the pressure data from the input port. Additionally, in other embodiments, the processing circuit receives the pressure data received from the input port as an analog signal providing continuous pressure data over time and converts the received pressure data into a digital signal.

Additionally, the control unit can be provided with an input for an accelerometer (e.g., pedometer, motion sensor), or a smart phone device, which includes an internal, built-in accelerometer. The accelerometer (or a smart phone device) can be configured to collect data relating to the orthosis wearer's gait and/or of the number of steps the orthosis wearer takes. This data is transferred to the control unit, processed by the processing circuit, and transmitted to the remote device. In this manner, the orthosis wearer's gait can be analyzed in conjunction with the pressure data. Additionally, the control unit can be configured to be responsive to the activity of the accelerometer. In this manner, when the orthosis wearer is not moving, as indicated by the accelerometer, the control unit will enter a power saving mode. The power saving mode can then be exited upon detecting subsequent movement, as also indicated by the accelerometer.

According to various aspects of the instant disclosure, certain embodiments of the orthosis pressure sensing device include multiple pressure sensor arrangements, having corresponding and respective pressure sensors, each of which can be configured to sense a different pressure range. Additionally, in these types of arrangements, the pressure sensor arrangements and/or the respective pressure sensors can have different shapes and sizes.

Aspects of the instant disclosure also relate to methods for sensing pressure between an orthosis and tissue of a patient. In such methods, the shape and the size of a patient wound are assessed. A particular pressure sensor is selected based on that assessment and upon the pressure sensors corresponding size and shape. The pressure sensor can then be fixed in a location that is proximal to the patient wound. Fixing the pressure sensor in this manner allows for the pressure between the orthosis and patient tissue to be sensed by the pressure sensor without irritating the patient tissue or wound. The sensor is communicatively coupled to a control unit. A wireless connection is established between the control unit and a software application running on a wireless communication device. Using the wireless connection, pressure data is communicated from the pressure sensor to the remote device using the software application. The software application can then provide feedback that is derived from the communicated pressure data. Additionally, a user interface is provided for storage, uploading and/or analysis of the communicated pressure data.

Various embodiments are directed toward different methods for store, displaying, reporting and communicating data from a pressure sensor system. This can include, but is not necessarily limited to, the use of printouts, emails, text messages and instant messages. Embodiments are directed toward the use of a configuration profile that specifies how information is stored and communicated. For instance, a treatment specialist can provide contact information, such as an email address and/or a telephone number. The system can provide the specialist with a list of different types of data and an option to select the different types of data. In response to selection, the system will communicate the associated data. Additional selection options can specify a desired method for how the selected type(s) of data are transmitted.

In various embodiments of the instant disclosure, a graph of pressure data over time can be generated and displayed using the software application. This software application can be provided on multiple remote devices such that the pressure data (and accelerometer data if an accelerometer is provided to the control unit) can be analyzed at multiple locations by multiple users. Additionally, because the control unit is WiFi enabled, the pressure (and accelerometer) data can be uploaded to a cloud-based service. In this manner, multiple remote users, each having access to a software application, can download and analyze the provided data from different locations over the Internet.

In various embodiments of the instant disclosure, a user interface provides options for selecting between modes of operation. The modes of operation can, for example, correspond to different types of physical activities. The data associated with the selected type of physical activity, as indicated by the selected mode, can also be stored with the corresponding pressure data. More particular examples are also possible including, but not necessarily limited to, those examples discussed herein. The control unit can be configured to operate according to any single one of the various modes or according to several different modes.

In other embodiments, the system can automatically detect the current operational mode. For instance, an accelerometer can be used in combination with a software application that includes an algorithm that detects sustained, high-level physical activity. For example, accelerometer data that is consistent with a sustained longer and quicker patient gait can be automatically detected and categorized as high-level physical activity. The corresponding pressure data can then be marked to indicate the high-level physical activity and a corresponding mode can be initiated.

Additionally, in certain embodiments of the instant disclosure, the software application may provide a training mode. In the training mode, feedback can be provided to the patient during physical activity, which may result in different levels of pressure between the patient and the orthosis. The feedback provides an indication of relative pressures occurring during the physical activity to facilitate improvement in use of the orthosis by the patient. When operating in training mode, the software application could, for example, detect pressure that exceeds a training threshold level and provide either an audible sound or visible cue to the patient. This can help the patient learn how to avoid actions that cause excessive pressure, which may cause problems in the future.

Consistent with embodiments of the present disclosure, a software application can be configured to apply an algorithm that identifies a patient's steps (e.g., heel strikes and/or toe strikes) by processing a waveform representing the pressure data. For instance, the algorithm can detect likely heel strikes by identifying (sharp) changes (increases) in pressure. The algorithm can further analyze the pressure data to detect periods during which neither the patient's limb nor the orthosis is not in contact with the ground. Thus, the algorithm can distinguish between weight shifting occurring during standing and weight changes caused by walking.

The identified steps can be correlated with additional information in the pressure data to assess the function of the orthosis and the use of the orthosis by the patient. In one particular example, the identified steps can be used to assess the patient's gait and to provide feedback so that the patient can improve upon their use of the orthosis (e.g., to reduce excessive pressure at critical areas and to prevent or lessen the development of wounds).

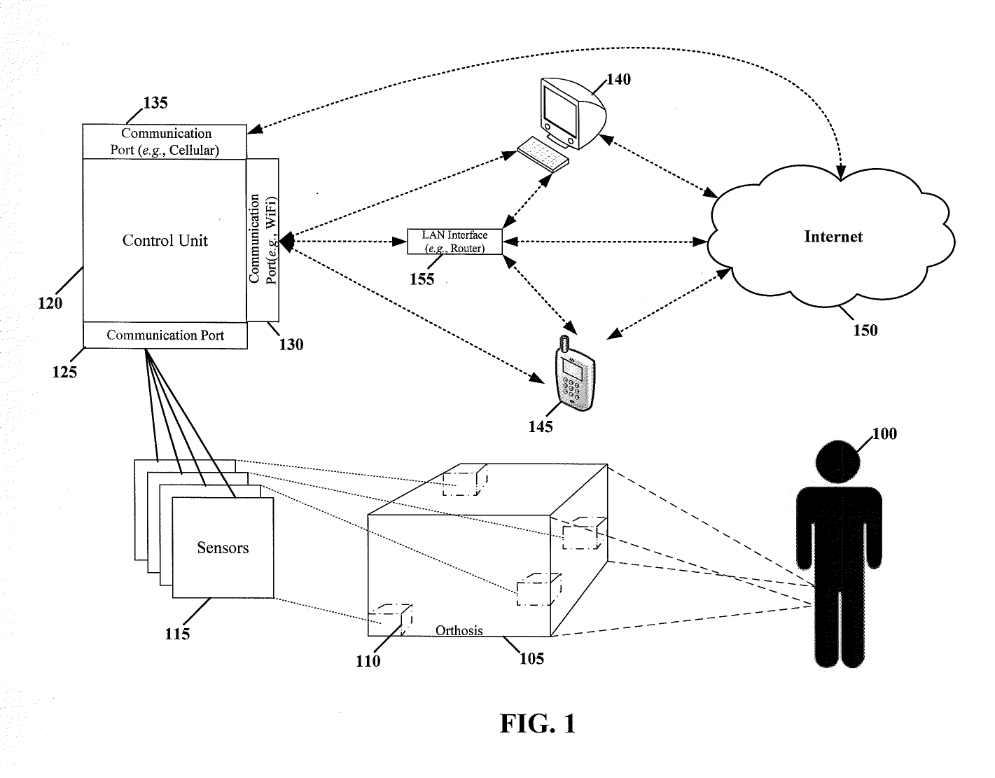

Turning now to the figures, FIG. 1 shows a diagram of an orthosis pressure sensing system as placed on a patient 100. The orthosis 105 is represented by a rectangular box to indicate that the orthosis 105 can be any number of different types of orthoses. Aspects of the present disclosure recognize that it can be advantageous to provide pressure sensors and associated systems that are designed for ease of use (and reuse) with many different types of orthoses. Several non-limiting examples for types of orthosis are provided herein.

One example type of orthosis is a medical device that is used to support and align the foot, to prevent or correct foot deformities, or to otherwise improve the functions of the foot. Such a foot orthosis can be designed to promote structural integrity of the joints of the foot and/or lower limb. For instance, a foot orthosis can counteract ground reaction forces, which can otherwise cause abnormal skeletal motion during a patient's gait (e.g., during the stance phase). A few examples of a foot orthosis include, but are not necessarily limited to, a sesamoid insert, a heel cup, or a University of California Biomechanics Laboratory (UCBL) Shoe Insert.

A shoe modification can be designed to encourage normal foot function by altering the magnitudes and temporal patterns of the reaction forces acting on the foot, and to decrease pathologic loading forces on the foot and lower limb during walking or other activities. A few non-limiting examples of shoe modifications include a cushioned heel, heel flare, heel wedge, extended heel or heel elevation rocker sole, metatarsal bar, sole wedge, sole flare or steel bar.

An orthosis can also be designed to support and align the joints of a lower limb, such as the knee or ankle. Such a device can be applied or attached to a lower limb to improve patient function. The device can stabilize the gait of a patient by providing necessary support. Pain can be reduced by transferring load from a problem location to another area. Deformities can be prevented, inhibited or corrected. A few non-limiting examples of ankle/foot orthoses (AFOs) include thermoplastic AFOs (posterior leaf spring [PLS], spiral AFO, hemi-spiral AFO, solid AFO, AFO with flange, hinged AFO, tone-reducing AFO [TRAF], free motion ankle joint; plantar flexion, dorsiflexion, and limited motion ankle joint stops; dorsiflexion assist spring joint; varus or valgus correction straps [T-straps]. A few examples of a knee orthoses (KOs) include orthoses for patellofemoral disorder (infrapatellar strap), for knee control in the sagittal plane, for knee control in the frontal plane and for axial rotation control.

Other orthoses can support the hip or trunk. Combinations of the above types of orthosis are also possible. Indeed, the sheer number of different orthoses can present a formidable challenge to achieving a proper fitting and to training and usage by the patient. Without being necessarily limited thereto, an orthosis 105 can be designed to facilitate patient's movement with stability and minimal energy output by encouraging a more normal gait. Achieving this goal can require both proper fitting of the orthosis 105 and adequate training for the patient. Accordingly, aspects of the present disclosure recognize that feedback on the effectiveness of the use of the orthosis can be particularly useful. Other aspects of the present disclosure can include, but are not necessarily limited to, an adaptive pressure sensing system that is designed for use with a variety of different orthoses and uses thereof.

The system of FIG. 1 can also include one or more pressure sensor(s) 115. The pressure sensors 115 convert sensed physical pressure into an electrical signal. The electrical signal is then provided to a control unit 120. The pressure sensors 115 can be designed for placement at an attachment area 110 between an orthosis 105 and the patient's skin with or without additional buffering or padding. In particular embodiments, the pressure sensors 115 are configured and arranged as a flat, primarily two-dimensional, shape. The thickness of the pressure sensors 115 can be kept low in order to facilitate their use and placement at virtually any location on the orthosis 105. This can be particularly useful for an orthosis 105 that has not been designed to accommodate the use of a pressure sensor. For instance, the relative thickness of the pressure sensors 115 can be thin enough to approximate the thickness for a liner material that might be used with the orthosis. A few examples of thicknesses include thicknesses of less than 5 mm, between 5 mm and 10 mm and thicknesses up to 15 mm. These example thicknesses, however, can be modified for different applications and do not limit all embodiments.

Consistent with embodiments of the present disclosure, pressure sensors 115 can also be designed to be attached to an orthosis 105 and/or the patient. For instance, the pressure sensors 115 can include an adhesive designed to adhere to a patient's skin and/or interface between the patient's skin and orthoses and to painlessly remove at a later time or designed with a strap for wrapping around a limb. In another instance, the pressure sensors can be fabricated directly into the orthoses and/or can include buttons, straps, adhesive, hooks or other connection solutions for attachment to the orthosis. Simple friction between the orthosis and patient can also be used. Using various fixation techniques and devices, the attachement locations 110 of the pressure sensors 115 can be set, changed (whether temporarily or permanently) as is represented by the dotted-lined boxes in the orthosis 105.

In one such embodiment, the pressure sensors 115 can include an adhesive that can be fixed to the orthosis 105 at a desired location. In certain instances, friction caused by pressure between the orthosis and the patient may be sufficient for attachment. Another, non-limiting embodiment uses a gel that helps hold the pressure sensors 115 in place. For instance, MED-6345 is a NuSil Technology product that functions as clear silicone tacky gel. This product is marketed as suitable for use in transdermal, wound-care, and hypertropic and keloid scar-management applications, among others. The gel provides temporary adhesive qualities that allow the product to adhere to the skin while enabling easy removal and reapplication. The pressure sensors 115 can be attached to such a gel, which provides adhesion to the skin of the patient (or to the orthosis 105).

In certain other embodiments, the orthosis 105 can be constructed to accommodate placement of one or more pressure sensors. A particular example is an orthosis 105 that includes one or more pockets that correspond to the size and shape of the pressure sensor. Other examples of attachment devices include a snap/button, Velcro and/or straps.

Consistent with certain embodiments of the present disclosure, the control unit 120 of the orthosis pressure sensing system is designed for the simultaneous connection to multiple pressure sensors 115. In this manner, the control unit 120 can collect pressure readings from multiple different locations in order to provide a more accurate indication of the effectiveness of the orthosis, the proper fitting and/or the patient's use of the orthosis. The system can be configured for use with different orthoses, patients, output data formats or the like, and thereby can provide a host of potential advantages and cost/time savings.

According to one embodiment of the present disclosure, the pressure sensors 115 utilize a standardized connector, which can be particularly useful for leveraging existing manufacturing and a user's familiarity with such standardized connectors. For instance, the connector can use a form factor that complies with one of the Universal Serial Bus (USB) standards.

In certain embodiments, the communication port(s) 125 can also include a USB protocol circuit for providing USB-compatible data communications to pressure sensors 115. The communication port 125 between the pressure sensors 115 and the control unit 120, however, does not necessarily need to use a USB-communication protocol. For instance, the pressure sensor 115 can provide an analog input using a USB connection circuit, which can be useful for limiting the costs, complexity, power draw and size of the pressure sensor 115 and control unit 120. In certain embodiments, for example, the pressure sensor 115 operates by modifying an electrical parameter (e.g., resistance, capacitance, voltage or current). For instance, the pressure sensors 115 can be made from a thin piezo electric film. The USB form factor provides several different electrical connection points that can be used to allow the control unit 120 to monitor and record such an electrical parameter directly (e.g., without first converting to a digital format for transmission over a USB protocol).

Communication port(s) 130/135 can be configured and arranged to allow the control unit 120 to connect to various different external devices and networks. For instance, the control unit 120 can communicate to a remote device 140/145 (e.g., smartphone, tablet or personal computer) using WiFi (e.g., IEEE 802.xx), Bluetooth or any other suitable wireless connection protocol. Depending on the functionalities available on the control unit 120, the wireless communication to the devices can piggyback on an existing wireless network router 155 (e.g., that is part of a local area network (LAN)). Alternatively or in addition, the control unit 120 can be configured and arranged to establish a direct communication link using, for example, a polling procedure or a scanning procedure.

Consistent with certain embodiments of the present disclosure, the control unit 120 is configured and arranged to detect the presence of a WiFi LAN and to attempt to connect thereto. The control unit 120 can be configured to detect the LAN automatically, based upon user-entered parameters or by way of first being directly connected to a wired port of the LAN (e.g., using a setup protocol such as the Wi-Fi Protected Setup (WPS)). Once connected to the LAN, the control unit 120 can attempt to find a remote device 140/145 that is running a specialized software application.

In a particular embodiment, the remote device 140/145 is a smartphone that has been configured with a downloadable software application. The smartphone is thereby configured to detect when a control unit 120 is available and to connect thereto. The connection protocol can allow any number of different a remote/wireless devices 145 running a software application (e.g., an iPhone, iPod touch, iPad, Android phone, touch pad or laptop) to connect to the control unit 120. For instance, a user can connect a pressure sensor to the control unit 120 before or after power is turned on. The control unit 120 can act as a wireless access point. The wireless device 145 can detect the wireless access point and connect thereto.

Particular embodiments recognize that the communications between the wireless device 145 and the control unit 120 can be implemented using a secure/encrypted connection. Other embodiments relate to limiting access to the control unit 120 to authorized users and devices. For instance, the control unit 120 can setup a wireless access point that is password protected. In certain embodiments, access to the control unit 120 is limited based upon whether or not a particular wireless device 145 has been licensed for use with the control unit 120. The licensing can be controlled by way of a software application that can enable or disable the connection between the wireless device 145 and the control unit 120. Moreover, the software application can limit the wireless device 145 to only certain control units 120 (e.g., through the use of device specific passwords).

Once a connection has been established, the smartphone and its software application can be configured to allow a user to access the control unit 120. This access can include, but is not necessarily limited to, various levels of control functions and the receipt of feedback based upon pressure sensor readings.

The wireless connection can be particularly useful in that it facilitates the placement of the smartphone in convenient locations. For instance, an orthopedic specialist can use their smartphone to control, monitor and assess a patient and the fitted orthosis while viewing the patient from an angle that allows the specialists to assess the patient's gait and to freely move into different positions or even provide physical support to the patient. In another instance, the patient can place their phone in a free hand, or other convenient location, in order to monitor or control the system while the patient is training or otherwise using the orthosis.

An orthosis wearer's gait may be different during different activities, such as during running versus walking. Therefore, selecting different modes of operation can indicate the differences between data collected during exercise and during normal wear. In another instance, the modes can indicate the aspects of the terrain. These aspects can include, as non-limiting examples, whether or not the wearer is indoors or outdoors; going up stairs, uphill or downhill; traversing obstacles or barriers; and ambulating on uneven terrain. The modes can also correspond to a patient's location, such as, at home, at work, in a vehicle, on a train, at a park or at a supermarket. As a function of the mode, warning indicators can be generated in response to the sensed pressure exceeding one or more limits, e.g., exceeding a maximum pressure threshold or inconsistently varying pressure, each of which might suggest an incorrect fit or improper usage. More intelligent analysis can detect that a pressure reading waveform has an unusual shape. This can include the detection of a rapid of a change in pressure (e.g., dP/dt), which may signify improper orthosis fit or patient gait.

The control unit can respond to different modes of operation by adjusting the sensor gathering parameters. For instance, the acceptable or expected pressure ranges can be set according to the particular mode and the associated activity. A mode for a leisurely stroll around the home may have a lower expected pressure range than a mode for a jog on a concrete sidewalk. Adjusting the expected pressure readings for different modes can be useful for improving the effective sensitivity of the collected data. For instance, the control unit may convert received pressure sensing data from an analog form to a digital form using an analog-to-digital converter (ADC) circuit with a certain resolution (e.g., 16 bits) and with a certain voltage (or current) input range (e.g., 0V-3V). The received pressure sensing data can be scaled such that the expected pressure ranges for the current mode fit within the voltage input range of the ADC circuit.

For instance, the expected voltage range for pressure readings in a first mode might be 0-0.5V and the control unit may have an ADC circuit with an input range of up to 3V. If an input voltage is outside of the ADC input range, the ADC may clip the signal and data can be lost. The control unit could therefore be configured to amplify the pressure readings by a multiple up to about 6 without clipping the pressure reading values within the expected range of 0-0.5V. The resolution of the ADC could then be applied across (nearly) the entire expected range (e.g., as opposed to having ADC bits reserved for sensor values outside of the expected range). In another mode the expected pressure reading values might be 0-4V. Some of the pressure reading values fall outside of the input range of the ADC and would therefore be clipped. The control unit could then be configured to scale the pressure reading values by a fractional amount and thereby fit the expected pressure reading values within the input range of the ADC.

The control unit can also be configured to target a specific range of pressure values in response to a selected mode. For instance, a pressure sensor may detect very little (or no) pressure until the patient's limb or othrosis contacts the ground. After contacting the ground, the pressure sensor may detect a significant increase in pressure. Generally speaking, as the range of sensed pressure values increases, the sensitivity of the stored readings decreases. The control unit can therefore be configured to respond to a particular mode by scaling and/or shifting the signal from the pressure sensor accordingly. As an example, the pressure might range from zero psi to near 40 psi during a normal gait. To closely analyze the pressure at the portion of the gait near 40 psi, the control unit can scale and shift the voltage such that the ADC circuit's operating input range corresponds to pressure readings centered on or near 40 psi. Low pressure readings, such as those near zero would therefore be effectively clipped. The use of modes for adjusting the effective sensitivity of the pressure readings can be particularly useful for providing improved sensitivity at a wide range of pressure values with relatively low resolution ADC circuits.

In more particular embodiments, the above scaling can be carried out dynamically to correspond to different portions within a patient's gait. In this manner, particular portions of the gait can be linked to an expected pressure range and the input to the ADC circuit can be adjusted accordingly.

Another parameter that can be adjusted includes the granularity of the collected data. For instance, high-impact activity may warrant a more precise set of pressure data over the period of activity than a low-impact activity. Accordingly, a mode for a high-impact activity can collect data at a high sample rate. A mode for a lower-impact activity may operate with a lower sample rate. In some modes, the controller may not actively provide pressure data until and unless a pressure limit is exceeded. Different sampling rates can be particularly useful for controlling the amount of data that is stored and eventually transmitted. Control of this variable can have a corresponding effect on the battery life of the control unit.

Yet another set of operational modes can correspond to the particular type of orthosis being used and in some instances, to the particular size and fitted settings. This can be particularly useful for adjusting the above mentioned parameters. Moreover, a particular embodiment is directed toward a control unit that is designed for use by a specialist. A specialist may use the same control unit for hundreds of different patients and their respective orthosis. The control unit can be configured with modes for different devices so as to facilitate the specialist's use of the device for each patient by adjusting the relevant sensing parameters. For instance, the expected range of pressures can be calibrated differently for each type of orthosis and respective placements of pressure sensors. The control unit can also be configured with knowledge of an expected number of pressure sensors for different types of orthosis. In certain embodiments of the present disclosure, the system is configured to provide a patient-by-patient mode. The specialist can setup a new mode for each patient. This mode can include information for the patient such as the type and configuration of the orthosis, the patient weight and pressure sensor calibration information. The specialist is thereby able to use, and easily recall, patient specific parameters for each subsequent session.

Consistent with embodiments of the present disclosure, the system can include a calibration mode, in which the system detects high and low pressure readings from a test run. These high/low pressure readings can be used as an expected range of the most and least amount of pressure during use of the orthosis. The received pressure sensing data can therefore be scaled such that the high pressure readings and low pressure readings fit within the voltage input range of the ADC circuit.

The system can be configured for use by a patient after having been fitted by a specialist. For instance, the system can be configured to monitor and record pressure readings over a period of hours, days, weeks or months. Such ongoing tracking can be facilitated by establishing a link between the control unit and a software application operating on a smartphone of the patient. The pressure readings can then be sent to the smartphone on a periodic or triggered/prompted basis. This data can then be provided to a specialist upon a patient's return visit by presenting the smartphone. The specialists can have a local control application, e.g., running on a computer or smartphone, that retrieves the stored information. In other embodiments, the smartphone application can upload the stored information to a specialist using the Internet. This can be particularly useful for allowing one or more specialists to remotely monitor the patient and their use of the orthosis.

Certain embodiments of the present disclosure are directed toward development of a patient-specific pressure reading baseline. This baseline can be developed while the patient is being monitored by the trained specialist. This helps to ensure that the baseline represents proper locomotion and gait. The baseline can be developed by combining a series of pressure readings taken while the patient is walking under supervision of the specialist. The system can provide an option for the specialist to discard one or more of the readings if the patient's gait is improper. The system can be configured to compute an average of the pressure readings and to generate the baseline from this averaging. For instance, the system can align the pressure readings into sets of readings corresponding to different phases of a gait, such as heel strike, foot flat, midstance, heel off, toe off, initial swing, midswing and terminal swing. This alignment allows for the pressure readings to be synchronized to each corresponding phase of the patient's gait and/or to each corresponding step. Thereafter, a patient can use the system to monitor their progress and to detect significant deviations from this baseline, which may indicate that the patient has regressed or otherwise have deviated from a desired gait and use of the orthosis. This data can be provided to the patient as feedback and/or to a treatment specialist to propose adjustment to the fitting of the orthosis and/or to the patient's use of the orthosis.

In certain embodiments, the different phases of a gait can be identified by the specialist. The pressure readings can be displayed as a waveform on a smartphone or computer. The specialist can set start and/or endpoints for each of the phases by manipulating the interface using an input device (e.g., a mouse, keyboard or touchscreen). In certain embodiments, the system is designed to operate in connection with a video camera. The video camera can be connected to an external device, such as a smartphone, laptop computer or similar processing device. The software application receives the pressure readings and correlates the timing of the pressure readings to the timing of the captured video data. A specialist can view the video (in slow or stop frame modes if desired) and then select the start and end of the different phases. The software application uses this information to define the corresponding time portions of the pressure reading data. In certain embodiments, the pressure reading data is overlaid with the video to allow viewing of both simultaneously.

In other embodiments, the software application can automatically assign different phases of a gait by analyzing the pressure reading data. The software application attempts to identify telltale signs of certain phases to provide a rough estimate of the location of the different phases. Further refinement can then be carried out using additional fitting algorithms that compare an expected pressure reading data with the actual pressure reading data. In certain embodiments of the present disclosure, the automated process is developed and improved by collecting pressure reading data from a significant number of patients and their respective gaits.

As an example, the software application can detect a large/fast pressure increase, followed by sustained pressure, as corresponding to a heel strike. From this data point, the rest of the phases can be ordered. The software algorithm can detect another heel strike to give an approximate timing for the remaining phases. The software algorithm can next look for more subtle indications of the locations of the remaining phases of the gait. This can include searching for pressure reading changes (Dp/dt) that suggest shifting of weight that corresponds to a transition between phases. It can also include searching for sustained pressure levels (within a set tolerance) for minimum time periods. This top-down approach can be particularly useful for relatively fast processing and for providing proper indexing of the pressure readings to the appropriate phase.

As another non-limiting example, the software application can attempt to match the pressure readings to one or more existing waveforms. The matching can be done using an appropriate waveform matching algorithm, such as least means squares or other error-based techniques. In particular embodiments, the software application makes use of waveform matching techniques used for high-speed data communications. For instance, filtering (low pass filtering, notch filtering or band pass filtering) can be used to remove unwanted signal components, which might be caused by electrical noise. For instance, a 60 Hz interference can be introduced from nearby AC power sources and individual electrical spikes can be produced from many different sources. Additionally, the waveform matching can make use of analysis in the time domain by the use of Fourier transforms. This can be particularly useful for detecting aspects of the waveform that are not necessarily otherwise easily identifiable to a human observer of the waveform and/or pressure readings data.

In certain embodiments of the present disclosure, a model of a system that includes a patient fitted with a particular type of orthosis can be developed and used to characterize subsequently received pressure readings. For instance, the model of the system is utilized to develop simulated waveforms. A matching is made between the voltage and current waveforms obtained by pressure sensor devices and those generated in simulations. The simulated waveforms can be compared with recorded ones (in the time domain and/or frequency domain), and the matching degree of the simulated and recorded waveforms is evaluated by using appropriate criteria. The various parameters of the model can then be modified according to certain approaches, and then the process repeats. The above steps are iterated until the simulated pressure waveforms best match the recorded pressure waveforms.

The existing waveforms can be developed by aggregation of many different patients and their corresponding orthoses. In many instances, the waveforms of different patients could exhibit certain characteristics that lend themselves to categorization. For instance, a first category of patients may exhibit an exaggerated hip, knee, or ankle flexion due to differences in leg lengths. In other instances, perceived joint instability may cause a patient to reduce the stance phase. Pain during gait can also result in slowing of the gait speed to attempt to reduce impact. These and other factors may result in measureably different pressure waveforms. Accordingly, the system is designed to receive waveforms from many different patients (and potentially many different treatment specialists) where the waveforms have been categorized with data regarding the patient's gait. Initially, the different categories can be manually identified by treatment specialists to develop a coherent database of waveform categories. Thereafter, a patient's pressure readings can be submitted and compared to the different waveform categories. This can be particularly useful for providing a diagnosis or recommendation for the patient. This can also be helpful to identify difficult-to-spot patient gait problems.

Certain aspects of the present disclosure are directed toward an embodiment of the system that tracks and reports usage to provide information to an insurance company. Insurance companies may offer discounts to patients who agree to participate in such reporting. The insurance company benefits from this monitoring capability to verify that the patient is following the proper treatment program, which can increase the likelihood of the patient's recovery and/or a positive outcome.

In addition to having the ability to collect and analyze data, these external devices 140/145 have the ability to provide feedback to the control unit 120, in order to make adjustments (e.g., data acquisition rate, on/off of sensors). The control unit 120 can establish and maintain connections to multiple devices 140/145, or a single device. Additionally, the control unit 120 can communicate the data acquired from the sensors to a WAN/Internet 155 enabled computer server. External users can therefore access the stored data, which can be analyzed using an appropriate device (e.g., smartphone, tablet, CPU).

Consistent with various embodiments of the present disclosure, one or more of the modes is controlled, initiated, selected and/or stopped using a software application running on an external device 140/145, such as a smartphone or computer. The control unit 120 can receive data indicating its operating parameters for the particular mode and from the external device 140/145. The selected modes can also be used to adjust the look of the interface that is provided to a user of the external device 140/145. For instance, a patient may select a training mode that provides feedback regarding desirable adjustments to a patient's gait. The interface can show a graphical image of the orthosis as well as where pressure is too high or too low (e.g., color coded lines on the graph correlating to pressure readings being acceptable, cautionary or to high). Further information can be provided regarding suggested reasons for the problematic pressure readings.

In certain embodiments, a software application operating on an external device 140/145 can provide a variety of different display options and types. For instance, a first display can provide instantaneous pressure readings using a needle-like gauge or digital read out. Another display can provide a graph of pressure readings overtime. The pressure readings can also be color coded according to the severity of the pressure (e.g., green for normal, yellow for caution and red for pressure). Certain embodiments provide multiple different graphs or gauges with different sensitivity and range of pressure values. For instance, a first graph can display pressure from 0 to 1 lbs and a second graph can display pressure from 0 to 2 lbs. Additional graphs and pressure ranges can be provided as desired. In a particular instance, a user can select between the different graph options. In other instances, the software application can automatically switch the graphical range to account for pressure changes (e.g., by setting graph ranges in response to the largest pressure reading being displayed). Other modifications and graph alternatives relate to different time scales.

In addition to (or in place of) visual/graphical indicators, the software application can also be configured to provide audible indications of the pressure readings. A few, non-limiting examples of audible indicators include a tone that changes in pitch depending upon the amount of pressure and an audible beep when a pressure limit has been exceeded.

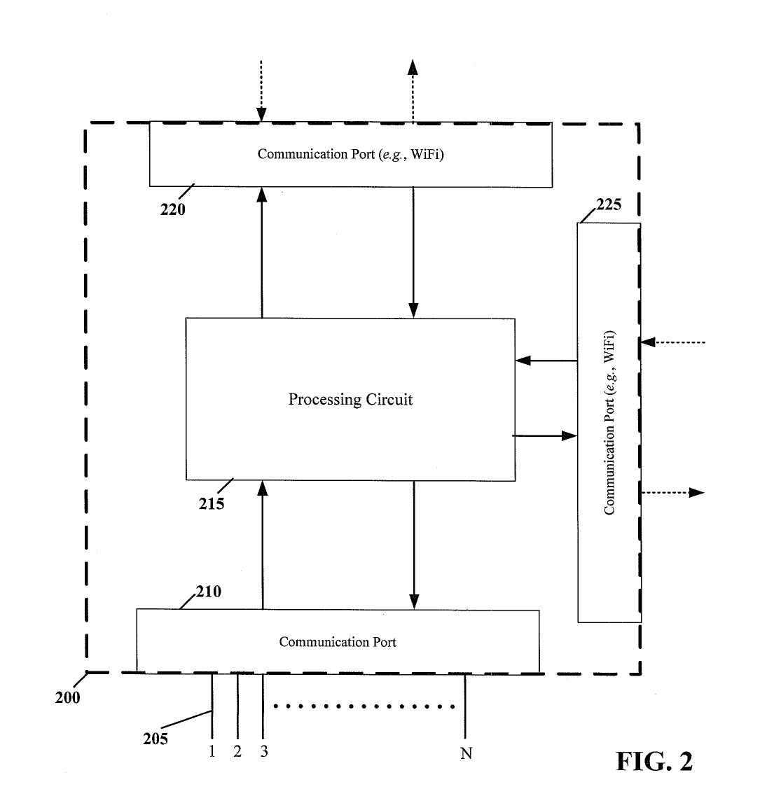

FIG. 2 shows a block diagram of the control unit, consistent with embodiments of the present disclosure. The control unit 200 contains a processing circuit 215 (e.g., microprocessor) that communicates with the pressure sensors 205. The processing circuit 215 can detect, for example, whether a sensor is present. The processing circuit 215 can also determine the number of sensors present and as measure and quantify the pressure sensed by the pressure sensor(s) 205. This connection is established through the communication port 210, which is further connected to the sensors 205 placed in the orthosis. The processing circuit 215 also can convert the pressure data into a wireless format, and communicate the data, for example, to a computer, smartphone, or to the Internet using a separate communications port(s) 220/225. Additionally, in response to communications made by the computer, smartphone, or the Internet, the processing circuit 215 can execute control functionalities (such as adjusting the data acquisition rate) relative to the pressure sensors 205. In certain embodiments, the control unit 200 can be powered using a rechargeable or replaceable battery.

The control unit 200 can be provided with a number of different interactive user tools. For example, the control unit 200 can have a graphic user interface. This user interface can show that the pressure sensor (or sensors) 205 is properly connected. The user interface can also provide information relating to the differing sensing modes, thus giving feedback to the orthosis wearer. The control unit 200 can also be provided with a simplified indicator (red/green/yellow LEDs) for noting whether the pressure sensor (or sensors) is connected correctly. The control unit 200 is connected to the pressure sensor arrangement(s) 205, which are placed in the orthosis. The control unit can be affixed to the body of the patient by using a belt clip, for example, or affixed to the orthosis. The control unit 200 can have a curved external body in order to blend well with the orthosis, and therefore minimize the control unit's obtrusiveness. The control unit 200 can also be fabricated directly into the orthosis.

In certain embodiments, the control unit 200 has an additional memory storage unit such that the pressure (and accelerometer) data are stored on-board with the control unit. In these instances, the control unit 200 includes logic circuitry that converts data to a USB compatible protocol for transmission (e.g., as opposed to only USB compatible connectors and wires). In one embodiment, a port can be connected to a USB compliant storage device (e.g., a thumb drive). Pressure reading data can be stored on this device, which can be detached and connected to a remote processing device (e.g., a computer). In certain instances, the data can be stored on the storage device at times when there is no active connection a wireless connection, to a smartphone, or other remote device. This can facilitate the ability to continuously monitor a patient, even in the absence of such a remote device.

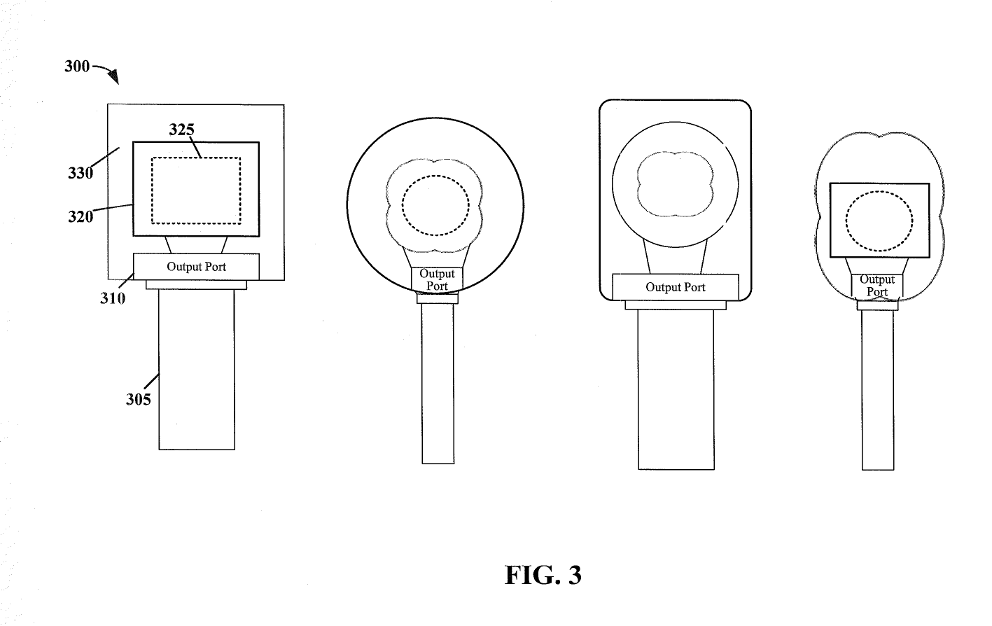

FIG. 3 shows a more detailed portrayal of multiple different example sensor arrangements 300. The sensors arrangements 300 can be connected to a control unit through using of a communication port or output port 310. The control unit can be connected to a number of different sensors using a single cable that can be quickly connected and disconnected from different sensors. Through a communication port, the control unit can be connected directly to a computer, to a smartphone, and/or to the Internet (or WAN). Further, the control unit can be connected through a network (e.g., LAN) to a computer, smartphone, and/or the Internet. The computer or smartphone can be equipped with a user interface such that the data provided from the pressure sensors can be viewed and analyzed. Further, the data transmitted to the Internet (or WAN) can be accessed and analyzed and stored to a file.

The pressure sensors arrangements 300 include a material 330 that surrounds a sensor 325 and can provide mechanical support as well as attachment mechanism(s). The material 330 that surrounds the sensor 325 is represented by the outermost solid-lined shape, whereas the active portion 320 of the sensor arrangement is the second solid-lined shape. As can be seen, the material 330 and the sensor 325 can have a number of different shapes and sizes depending on the application. The material 330 can be pliable (or formable). Further, as represented by the dotted-lined shapes, the sensors (and/or the surrounding material) can have “cut-outs” 325. These “cut-outs” 325 are provided so the sensor arrangements can determine the pressure around a wound in an orthosis without actually coming into contact with the wound. The “cut-outs” 325 are available in multiple shapes and sizes that can be selected based upon the particular needs of the patient and treating specialist. The shapes of the sensor arrangements 300 and illustrated sensors 325 are not limiting. For instance, the shape of the active portion 320 and the sensor arrangement shapes 300 are interchangeable (e.g., a square shaped sensor can also be provided with a circular sensor arrangement). Additionally, the shape of the active portions 320 and cut-outs 325 are interchangeable with the sensor arrangements 300. Further, each part can be more specifically tailored based on a patient's assessed wound or injury.

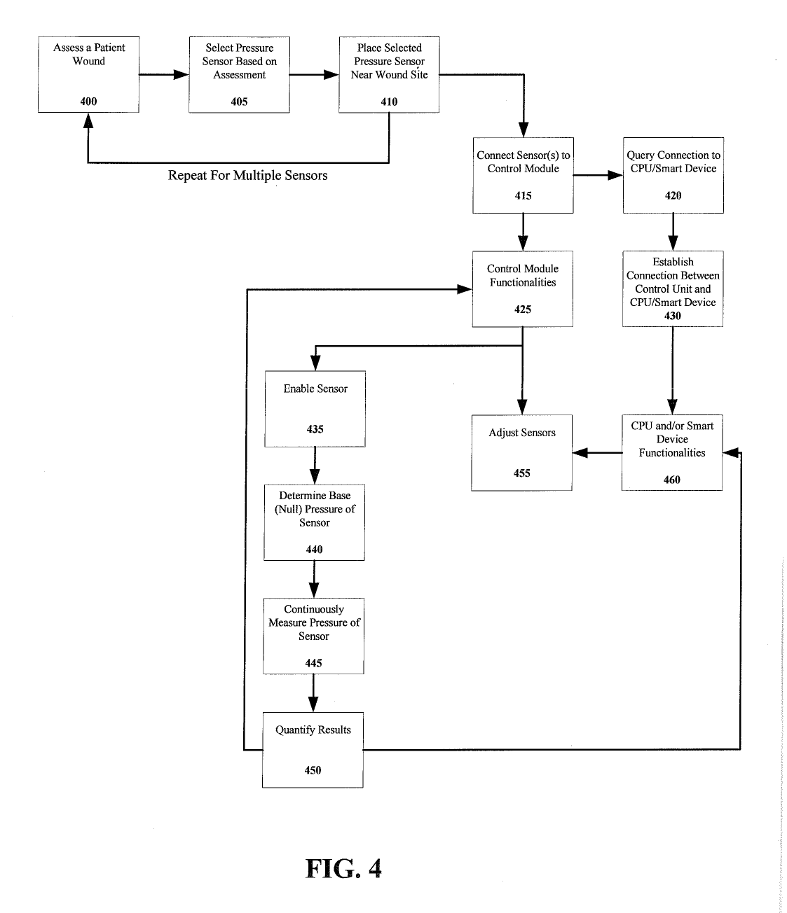

FIG. 4 shows an example flowchart of an operation of an orthosis pressure sensing system. As shown in block 400, a patient is first assessed to determine an appropriate orthosis and sensor arrangement. Based on that assessment, a pressure sensor is selected 405, and placed near the wound site 410. This can be repeated for multiple sensor arrangements. The sensor(s) is connected to a control module 415. The control unit will query to determine if it is currently connected to a computer, smartphone, the Internet, or multiple connections 420 and/or to determine whether any additional connections are desirable. Depending on the application, the control unit can make the connection to the desired peripherals 430. The control unit will enable the sensor 435 (or sensors) and perform a calibration procedure, such as determining the base (null) pressure of each of the sensors 440. The sensing circuits (e.g., analog scaling for an ADC input) of the control unit can thereafter be set according to the calibration procedure. The pressure sensors can thereafter be continuously monitored 445, and the control unit (and/or peripherals) will collect the data. Additionally, the control unit, and/or the peripherals can quantify and summarize the results into a more readily understood form 450. The control unit, and/or the peripherals, can be configured to calibrate and adjust in response to different sensors as they are connected and/or to different placements and uses of the sensors 455. Further, based on the functionalities provided to the control module, the data rate (samples/second) of data retrieved from the pressure sensors can be adjusted 425.

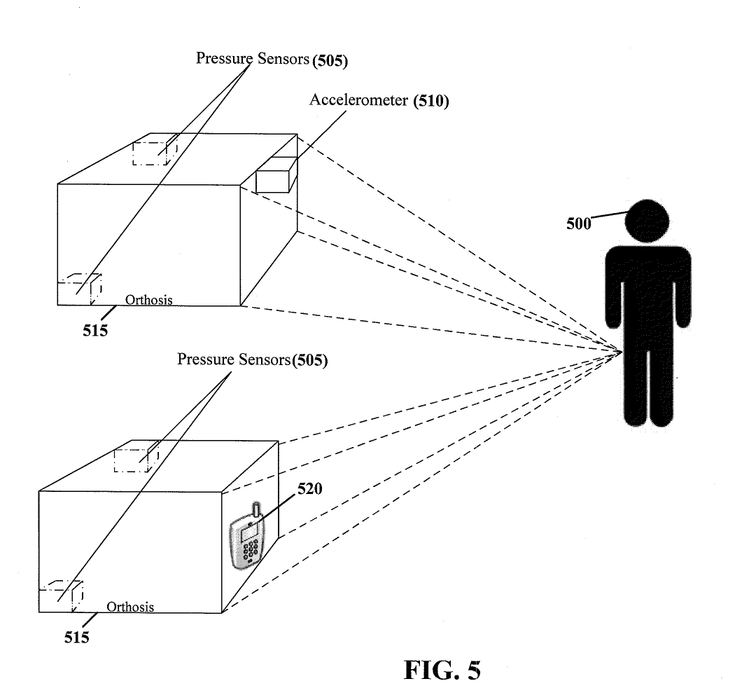

FIG. 5 shows placement of a smartphone 520 to operate as an accelerometer (or pedometer), as well as placement of an accelerometer 510 with the orthosis arrangement. The accelerometer 510 and the smartphone 520 are shown as placed on the orthosis 515, as well as the placements of pressure sensors 505 within the orthosis 515. In certain embodiments, the smartphone 520 functionalities shown in the flow diagram can include accelerometer-related capabilities. For instance, a smartphone's existing accelerometer and related sensors can be programmed to provide additional data that can be useful for detecting steps or other phases of a gait. Consistent with certain embodiments, an accelerometer 510 can be incorporated into the orthosis arrangement or directly into the control unit. Such additional data relates to a person's gait and movement, and can then be used to quantify and compare that against the pressure sensor data.

In another instance, an accelerometer can be used to detect potential heel strikes by monitoring the occurrence of significant acceleration events. Additional data can be obtained from more subtle acceleration events, such as the pause and reversal of direction occurring at the leg swing transition between forward and backward motion.

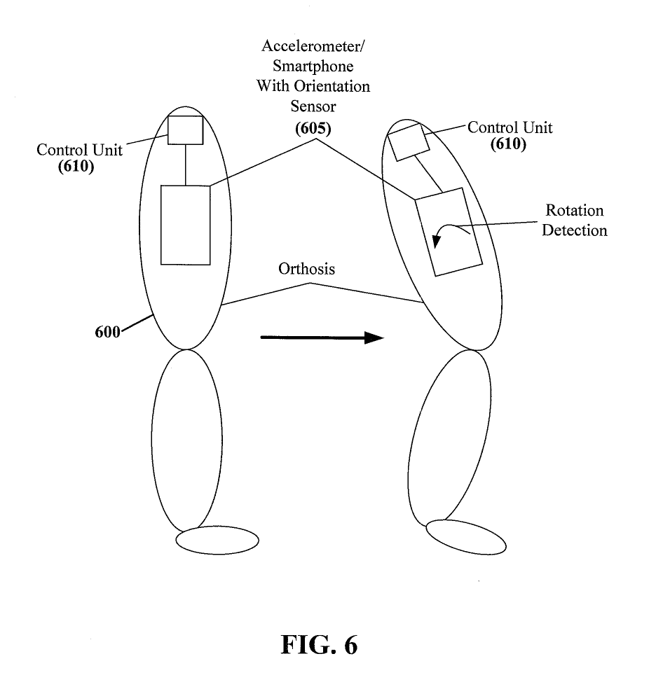

FIG. 6 shows a device 605 (e.g., a smartphone) placed on an orthosis 600 for measuring orientation changes during the gait of a patient wearing the orthosis 600. The device 605 is shown connected to a control unit 610, which collects and quantifies the data from the device 605. The control unit 610 can be worn on the patient's hip or other appropriate place on the body or be fabricated directly into the orthosis. The pressure sensors are not shown in FIG. 6, but are connected from the control unit 610 and in the interior of the orthosis 600. The smartphone 605 can measure information about the gait of the patient by monitoring the angle at which the leg is changed during different portions of the gait.

For instance, many smartphones contain rotational information that determines the smartphone's orientation using a gravitational-based sensor(s). In this manner, the smartphone can perform operations such as rotating the display according to the user's current viewing preference. Aspects of the present disclosure use this rotational data to further process the pressure reading data. For instance, the smartphone can be placed parallel to the femur. The femur swings through different orientations which can be correlated to the rotational data retrieved from the smartphone sensor. This information is then linked to associated gait phases and to corresponding pressure readings from a corresponding time period.

Other aspects of the present disclosure contemplate the use of an accelerometer and/or rotational sensor on each lower limb. In this manner, additional data points can be collected to further enhance waveform processing. For instance, a control unit could contain a first accelerometer (or rotational) sensor and a smartphone could contain a second accelerometer (or rotational) sensor. The control unit could be attached to a first lower limb, while the smartphone is attached to a second. The data from each of the sensors could be used to further define the gait phases. Moreover, asymmetry in the patient's gait can be detected by comparing the data from each of the multiple sensor devices.

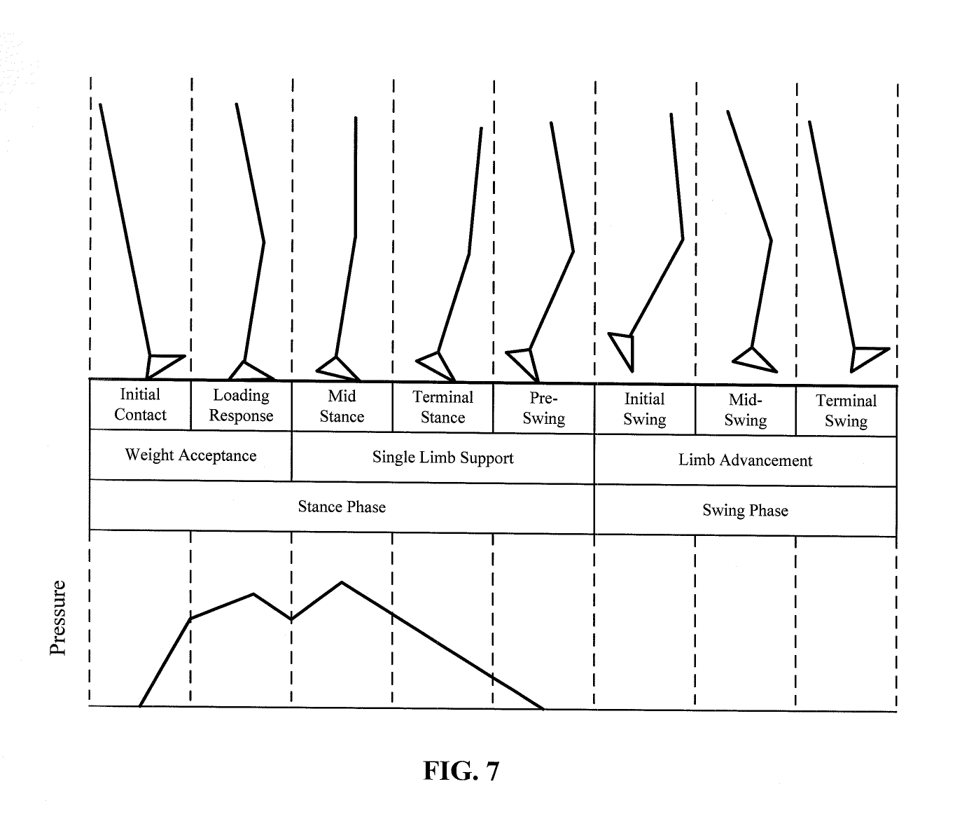

FIG. 7 depicts a plurality of different gait phases and a corresponding pressure waveform, consistent with embodiments of the present disclosure. As shown in FIG. 7, the gait phases are not limited to any particular breakdown and can be adjusted as necessary. For instance, a relative broad set of gait phases includes just two phases for the entire gait-stand phase and swing phase. Another, more specific, set of gait phases includes weight acceptance, single limb support and limb advancement. Moreover, the set of gait phases can be more particularly broken apart into initial contact, loading response, midstance, terminal stance, pre-swing, initial swing, mid-swing, and terminal swing.

As the gait phases become more specific, the accuracy of the waveform correlation may become lessened. Accordingly, embodiments of the present disclosure can use a tiered gait phase breakdown from general to more specific. Moreover, aspects of the present disclosure include a confidence factor that can be determined based upon the error between the pressure readings and the expected waveforms for each gait phase. This can be particularly useful for helping a patient or specialist assess the waveform data and potentially to disregard data having a low confidence level.

The signals and associated logic and functionality described in connection with the figures can be implemented in a number of different ways. Unless otherwise indicated, various general-purpose systems and/or logic circuitry may be used with programs in accordance with the teachings herein, or it may prove convenient to use a more specialized apparatus to perform the disclosed aspects. For example, according to the present disclosure, one or more of the methods can be implemented in hard-wired circuitry by programming a general-purpose processor, other fully or semi-programmable logic circuitry, and/or by a combination of such hardware and a general-purpose processor configured with software. Accordingly, the various components and processes shown in the figures can be implemented in a variety of circuit-based forms, such as through the use of data processing circuit modules.

It is recognized that aspects of the disclosure can be practiced with computer/processor-based system configurations other than those expressly described herein. The required structure for a variety of these systems and circuits would be apparent from the intended application and the above description.

The various terms and techniques are used by those knowledgeable in the art to describe aspects relating to one or more of communications, protocols, applications, implementations, and mechanisms. One such technique is the description of an implementation of a technique expressed in terms of an algorithm or mathematical expression. While such techniques may be implemented, for example, by executing code on a computer, the expression of that technique may be conveyed and communicated as a formula, algorithm, or mathematical expression.

For example, a block or module denoting “C=A+B” as an additive function implemented in hardware and/or software would take two inputs (A and B) and produce a summation output (C), such as in combinatorial logic circuitry. Thus, the use of a formula, algorithm, or mathematical expression as descriptions is to be understood as having a physical embodiment in at least hardware (such as a processor in which the techniques of the present disclosure may be practiced as well as implemented as an embodiment).

In certain embodiments, machine-executable instructions are stored for execution in a manner consistent with one or more of the methods of the present disclosure. The instructions can be used to cause a general-purpose or special-purpose processor that is programmed with the instructions to perform the steps of the methods. The steps may be performed by specific hardware components that contain hardwired logic for performing the steps, or by any combination of programmed computer components and custom hardware components.

In some embodiments, aspects of the present disclosure may be provided as a computer program product, which may include a machine or computer-readable medium having stored thereon instructions, which may be used to program a computer (or other electronic devices) to perform a process according to the present disclosure. Accordingly, the computer-readable medium includes any type of media/machine-readable medium suitable for storing electronic instructions.

Various modules may be implemented to carry out one or more of the operations and activities described herein and/or shown in the figures. In these contexts, a “module” is a circuit that carries out one or more of these or related operations/activities. For example, in certain of the above-discussed embodiments, one or more modules are discrete logic circuits or programmable logic circuits configured and arranged for implementing these operations/activities, as in the circuit modules shown in FIGS. 1-7. In certain embodiments, the programmable circuit is one (or more) computer circuits programmed to execute a set (or sets) of instructions (and/or configuration data). The instructions (and/or configuration data) can be in the form of firmware or software stored in and accessible from a memory (circuit). As an example, first and second modules include a combination of a CPU hardware-based circuit and a set of instructions in the form of firmware, where the first module includes a first CPU hardware circuit with one set of instructions and the second module includes a second CPU hardware circuit with another set of instructions.

Aspects of the present invention are implemented using a variety of processing circuits, logic, communications arrangements and combinations thereof. Particular implementations use one or more specially configured computer processors that execute instructions to perform one or more of the aspects discussed herein. Various portions can be implemented using discrete or combinatorial logic, analog circuitry and using various forms of tangible storage mediums.

The various embodiments described above are provided by way of illustration only and should not be construed to limit the invention. Based upon the above discussion and illustrations, those skilled in the art will readily recognize that various modifications and changes may be made to the present invention without strictly following the exemplary embodiments and applications illustrated and described herein. For example, the methods, devices and systems discussed herein may be implemented in connection with a variety of technologies such as those involving home computers, servers, laptops, cellular phones, personal digital assistants, iPhones®, Blackberries® and the like. The invention may also be implemented using a variety of approaches such as those involving coordinated communications for public access. Such modifications and changes do not depart from the true spirit and scope of the present invention, including that set forth in the following claims.

1. An orthosis pressure sensing apparatus comprising:
a control unit that includes
an input port configured and arranged to receive pressure data from pressure sensor arrangements;
a wireless interface circuit configured and arranged to communicate with remote devices using wireless communications;
a processing circuit configured and arranged to
detect a wireless device running a software application, the detection responsive to data received from the wireless interface circuit;
establish communications with the detected wireless device by exchanging data with the software application using the wireless interface circuit;
format pressure data received from the input port according to a wireless transmission protocol used by the software application; and
transmit, using the wireless interface circuit, the formatted pressure data to the software application; and
one or more pressure sensor arrangements, each arrangement including
an output port that includes a connector that is designed for insertion into and removal from the input port and that is designed to create a communication link to the input port when inserted, and thereby facilitate the connection of different pressure sensor arrangements to the same control unit;
a pressure sensor designed for selective placement at multiple locations of interest that include different contact points between an orthosis and a patient; and
a communication line configured and arranged to carry the pressure data from the pressure sensor to the output port.

2. The apparatus of claim 1, wherein the input port is configured and arranged for hot swapping of the one or more pressure sensor arrangements.

3. The apparatus of claim 1, wherein the one or more pressure sensor arrangements include multiple pressure sensor arrangements, each pressure sensor arrangement having a corresponding and respective pressure sensor configured and arranged with different pressure sensing patterns, whereby the different pressure sensing patterns can be selected relative to a size and shape for a patient wound.

4. The apparatus of claim 1, wherein the pressure sensor has a pattern that is both designed to sense pressure between the orthosis and patient tissue surrounding a patient wound and has an opening to reduce or avoid contact with a patient wound.

5. The apparatus of claim 1, wherein the one or more pressure sensor arrangements include an attachment mechanism for attaching the pressure sensor to each of multiple different locations, the locations being relative to the orthosis as fitted to a patient.

6-7. (canceled)

8. The apparatus of claim 1, wherein the one or more pressure sensor arrangements include multiple pressure sensor arrangements, each pressure sensor arrangement including corresponding and respective pressure sensors, each pressure sensor having a different pressure range.

9. The apparatus of claim 1, further including a motion sensor configured and arranged to provide motion data indicative of steps taken by a patient.

10. The apparatus of claim 9, wherein the processing circuit is further configured and arranged to identify steps taken by a patient in response to the motion data and to transmit an indication of a correlation between the pressure data to the software application.

11. The apparatus of claim 9, wherein the control unit is configured and arranged to operate in a training mode that is designed to detect pressure that exceeds a training threshold level and notify the patient using either an audible sound or visible cue.

12. The apparatus of claim 1, wherein the processing circuit is further configured and arranged to perform the formatting of the pressure data received from the input port and the transmitting of the formatted pressure data in real time, relative to receipt of the pressure data from the input port.

13. The apparatus of claim 1, wherein the processing circuit is further configured and arranged to receive the pressure data received from the input port in as an analog signal providing continuous pressure data over time and to convert the received pressure data into a digital signal.

14. A method for sensing pressure between an orthosis and tissue of a patient, the method comprising:
assessing a shape and size of a patient wound;
selecting a pressure sensor based upon a correlation between a pattern of active pressure sensing area for the pressure sensor and the assessed shape and size of a patient wound;
fixing the pressure sensor in a location proximal to the patient wound to allow pressure between the orthosis and patient tissue to be sensed by the pressure sensor;
communicatively coupling the pressure sensor to a control unit;
establishing a wireless connection between the control unit and a software application running on a wireless communication device;
communicating pressure data received from the pressure sensor to the software application using the wireless connection;
providing, using the software application, feedback indicative of the communicated pressure data; and
providing a user interface having options for storage and uploading of the communicated pressure data.

15. The method of claim 14, further including the steps of generating and displaying, using the software application, at least one of a graph of pressure data over time, an instantaneous pressure reading from a needle gauge and a digital readout.

16. The method of claim 14, further including the steps of
generating a patient baseline for pressure sensor readings,
detecting a deviation from the patient baseline, and
providing an indication of the deviation to the patient.

17. The method of claim 14, further including the steps of
providing the user interface with options for selecting between modes of operation that correspond to different types of physical activities;
storing data associating a type of physical activity indicated by a selected mode with corresponding pressure data; and
providing an interface for reviewing the pressure data relative to the associated type of physical activity.

18. The method of claim 14, further including the steps of
initiating a training mode using the software application; and
providing feedback to the patient during a physical activity that results in different pressures between the patient and the orthosis, wherein the feedback provides an indication of relative pressures occurring during the physical activity to facilitate improvement in use of the orthosis by the patient.

19. The method of claim 14, further including the steps of
applying, using the software application, an algorithm that identifies steps in a gait of the patient by processing a waveform representing the pressured data; and
align the pressure data into sets of readings corresponding to different phases an identified step of the gait; and
providing an indication of improper gait based upon steps identified by the algorithm and the associated sets of readings.

20. The method of claim 14, further including the steps of
collecting data relating to an orthosis wearer's gait from an accelerometer device;
analyzing the collected data to identify sustained longer and quicker patient gait that is consistent high-level physical activity; and
initiating, in response to identifying high-level physical activity, a corresponding mode for the software application in which corresponding pressure data is marked as high-level physical activity.

21. The method of claim 14, further including the steps of
storing pressure readings to a patient's smartphone,
presenting the smartphone to another processing device, and
retrieving, using a control application running on the processing device, the stored pressure readings from the patient's smartphone.

22. The method of claim 14, further including the steps of
detecting an absence of a wireless connection with the control unit,
storing, in response to detecting the absence of the wireless connection, pressure sensor data in the control unit,
detecting a subsequent wireless connection with the control unit, and
uploading, in response to detecting the wireless connection, the stored pressure sensor data from the control unit.

23. The apparatus of claim 1, wherein the connector has a form factor that complies with a USB standard and wherein the pressure sensor arrangement is configured and arranged to communicate the data from pressure sensor to the control unit in an analog form.

24. The apparatus of claim 1, further including the wireless device running the software application and wherein the software application is configured and arranged to enable and disable the connection between the wireless device and the control unit in response to determining whether a license limits use of the apparatus.
